(19) United States
(12) Patent Application Publication
(10) Pub. No.: US 2010/0010550 A1
(43) Pub. Date: Jan. 14, 2010

(54) BRANCHING THERAPY ELEMENTS AND METHOD OF THEIR INSERTION INTO LIVING TISSUE

(75) Inventors: Youri Ponomarev, Leuven (BE); Matthias Merz, Leuven (BE); Remco Henricus Wilhelmus Pijnenburg, Hoogeloon (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/442,528

(22) PCT Filed: Sep. 20, 2007
(86) PCT No.: PCT/IB07/53824

§ 371 Date: Mar. 24, 2009

Related U.S. Application Data
(60) Provisional application No. 60/826,773, filed on Sep. 25, 2006.

(51) Int. Cl. A61N 1/05; A61M 31/00; A61B 19/00

(52) U.S. Cl. 607/3; 607/2; 606/129

(57) ABSTRACT

An implantable medical system for electrical recording and or providing therapy to a plurality of tissue sites without damage to surrounding blood vessels is disclosed comprising: an implant body having a plurality of therapy elements, the elements being hingedly attached at one end to the surface of the body and releasably extendable outward from the surface of the body at the other end; a release mechanism for each of the elements; and a coating material covering the body and the elements; wherein upon dissolution of the coating material after implantation, the release mechanism is capable of causing the elements to extend outward at one end from the surface of the body and into a plurality of tissue sites without damage to the surrounding blood vessels. The method of implanting the system into a body is also disclosed.

The disclosure is directed to an implantable medical system for electrical recording and or providing therapy, such as for example drug delivery or stimulation of a plurality of tissue sites, such as neural tissue sites, without damage to surrounding blood vessels in a human or animal body.

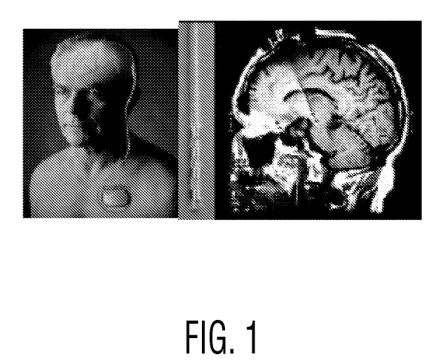

In recent years electrical recording and or stimulation of the nervous tissue has been successfully used in treating symptoms of neural diseases (for example, Parkinson's or epilepsy). The efficiency of the treatment is determined by the precision of the electrode placement and, so far, has been limited to positioning multi-site cylindrical metal structures (see, for example, FIG. 1 which depicts the Medtronic™ DBS electrodes for treating Parkinson's disease).

During neurosurgical procedures, electrodes are commonly used to monitor electrical activity and/or to stimulate neural tissue. Neurostimulation systems may be used to deliver neurostimulation therapy to patients to treat a variety of symptoms or conditions such as chronic pain, tremor, Parkinson's disease, multiple sclerosis, spinal cord injury, cerebral palsy, amyotrophic lateral sclerosis, dystonia, torticollis, epilepsy, incontinence, or gastroparesis. A neurostimulation system delivers neurostimulation therapy in the form of electrical pulses. In general, neurostimulation systems deliver neurostimulation therapy via electrodes included in an implantable body or stimulation lead, which is located proximate to the neural tissue sites of interest such as spinal cord, pelvic nerves, pudendal nerve, or stomach, or within the brain of a patient. The stimulation leads may include percutaneously implanted leads or surgically implanted leads. Such stimulation systems, including neurostimulation systems, are disclosed in U.S. Patent Application Publications 2005/0096718 published on May 5, 2005, 2004/0186544 published on Sep. 23, 2004, 2004/0186543 published on Sep. 23, 2004, 2004/0015221 published on Jan. 22, 2004, 2003/0114905 published on Jun. 19, 2003, 2003/0176905 published on Sep. 18, 2003 and 2003/0083724 published on May 1, 2003.

Recent efforts in the medical field have focused on the delivery of therapy, not only in the form of electrical stimulation, but also in the delivery of drugs to precise locations within the human body. Therapy originates from an implanted source device, which may be an electrical pulse generator, in the case of electrical therapy, or a drug pump, in the case of drug therapy. Therapy is applied through one or more implanted leads that communicate with the source device and include one or more therapy delivery sites for delivering therapy to precise locations within the body.

In drug therapy systems, delivery sites take the form of one or more catheters. In electrical therapy systems, they take the form of one or more electrodes wired to the source device. In Spinal Cord Simulation (SCS) techniques, for example, electrical stimulation is provided to precise locations near the human spinal cord through a lead that is usually deployed in the epidural space of the spinal cord. Such techniques have proven effective in treating or managing disease and acute and chronic pain conditions. Such drug therapy is disclosed for example in U.S. Patent Application Publications 2004/0186543 published on Sep. 23, 2004 and 2003/0083724 published on May 1, 2003.

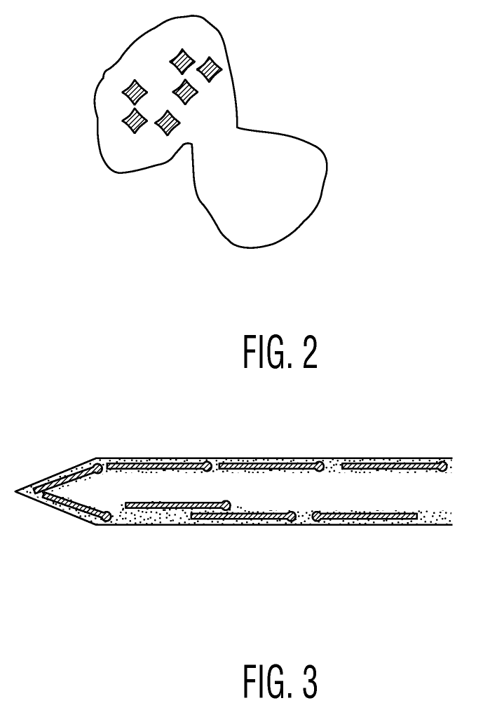

It is desirable, however, to record activity and/or provide therapy, such as drug delivery or stimulate certain parts of the brain or any other electrogenic tissue at different spots, close to each other, but not necessarily at the same time (see FIG. 2). This is currently not possible, since it would require extremely complex multiple implantations of electrodes with a large probability of tissue damage and other post-operative complications. It is especially difficult to put the electrodes into the right positions of the tissue sites of interest without severing the blood vessels in the surrounding area.

These and other needs are satisfied with the system and method of the present disclosure.

According to the present disclosure, an implantable medical system for electrical recording and or providing therapy to a plurality of tissue sites without damage to surrounding blood vessels in a human or animal body is disclosed, as well as the method of implanting the system into a human or animal body.

Specifically it is an object of the invention to provide an implantable medical system for providing electrical recording and or therapy to one or more tissue sites of a mammal without damage to surrounding blood vessels comprising:
an implant body having at least one therapy element, each element being hingedly attached at one end to the surface of the body and releasably extendable outward from the surface of the body at the other end;
a release mechanism for each element; and
a coating material covering the body and each element; wherein upon dissolution of the coating material after implantation, the release mechanism is capable of causing each of the elements to extend outward at one end from the surface of the body and into the one or more tissue sites without damage to the surrounding blood vessels.

Another object is to provide a system wherein at least one of the therapy elements is capable of delivering a drug to the one or more tissue sites.

Another object is to provide a system wherein the coating material is frozen water.

Another object is to provide an implantable electrode system for electrical recording and or stimulation of a plurality of neural tissue sites without damage to surrounding blood vessels comprising:
an implant body having a plurality of electrodes, the electrodes being hingedly attached at one end to the surface of the body and releasably extendable outward from the surface of the body at the other end;
a release mechanism for each of the electrodes; and
a biodegradable coating material covering the body and the electrodes; wherein upon dissolution of the coating material after implantation, the release mechanism is capable of causing the electrodes to extend outward at one end from the surface of the body and into a plurality of neural tissue sites without damage to the surrounding blood vessels.

Another object is to provide a system wherein the release mechanism comprises a stress coating material on a portion of the outer surface of the electrode, the stress coating material having a lower Young's modulus value than that of the electrode; and the biodegradable coating material covers the body and the stress coated electrodes.

Another object is to provide a system wherein the implant body is made of silicon.

Another object is to provide a system wherein the biodegradable coating material is poly(dl-lactide-co-glycolide) polymer which degrades by hydrolysis.

Another object is to provide a system wherein the electrodes are made of silicon and the stress coating material is gold.

Another object is to provide a method of implanting an implantable medical system for electrical recording and or providing therapy to one or more tissue sites without damage to surrounding blood vessels, the method comprising:
implanting the system into a desired location having the tissue sites, the system comprising:
an implant body having at least one therapy element, the element being hingedly attached at one end to the surface of the body and releasably extendable outward from the surface of the body at the other end;
a release mechanism for each element; and
a coating material covering the body and each element; wherein upon dissolution of the coating material after implantation, the release mechanism is capable of causing each of the elements to extend outward at one end from the surface of the body and into one or more tissue sites;
and activating the release mechanism thereby causing each of the elements to extend outwardly at one end from the surface of the body and into the one or more tissue sites without damage to the surrounding blood vessels.
Another object is to provide a method wherein at least one of the therapy elements is capable of delivering a drug to the one or more tissue sites.
Another object is to provide a method wherein the coating material is frozen water.

Another object is to provide a method of implanting an implantable electrode system for electrical recording and or stimulation of a plurality of neural tissue sites without damage to surrounding blood vessels, the method comprising:
implanting the system into a desired location having the neural tissue sites, the system comprising:
an implant body having a plurality of electrodes, the electrodes being hingedly attached at one end to the surface of the body and releasably extendable outward from the surface of the body at the other end;
a release mechanism for each of the electrodes; and
a biodegradable coating material covering the body and the electrodes; wherein upon dissolution of the coating material after implantation, the release mechanism is capable of causing the electrodes to extend outward at one end from the surface of the body and into a plurality of neural tissue sites;
and activating the release mechanism thereby causing each of the electrodes to extend outwardly at one end from the surface of the body and into the plurality of neural tissue sites without damage to the surrounding blood vessels.

Another object is to provide a method wherein the release mechanism comprises a stress coating material on a portion of the outer surface of the electrode, the stress coating material having a lower Young's modulus value than that of the electrode; and
the biodegradable coating material covers the body and the stress coated electrodes.

Another object is to provide a method wherein the implant body is made of silicon.

Another object is to provide a method wherein the biodegradable coating material is poly(dl-lactide-co-glycolide) polymer which degrades by hydrolysis.

Another object is to provide a method wherein the electrodes are made of silicon and the stress coating material is gold.

These and other aspects of the invention are explained in more detail with reference to the following embodiments and with reference to the figures.

FIG. 1 is a photograph depicting the use of the prior art Medtronic™ DBS electrode on the human head. The DBS electrode has four platinum/iridium contacts. For treating Parkinson's disease by neurostimulation, two electrodes are used to stop tremors on both the left and right sides of the body.

FIG. 2 is a sketch depicting a nervous tissue part (for example, subthalamic nucleus used to treat Parkinson's patients by using the DBS electrode) and desired neural tissue sites where recording and stimulation are performed.

FIG. 3 is a sketch depicting the implantable electrode system according to the invention before implantation into the body.

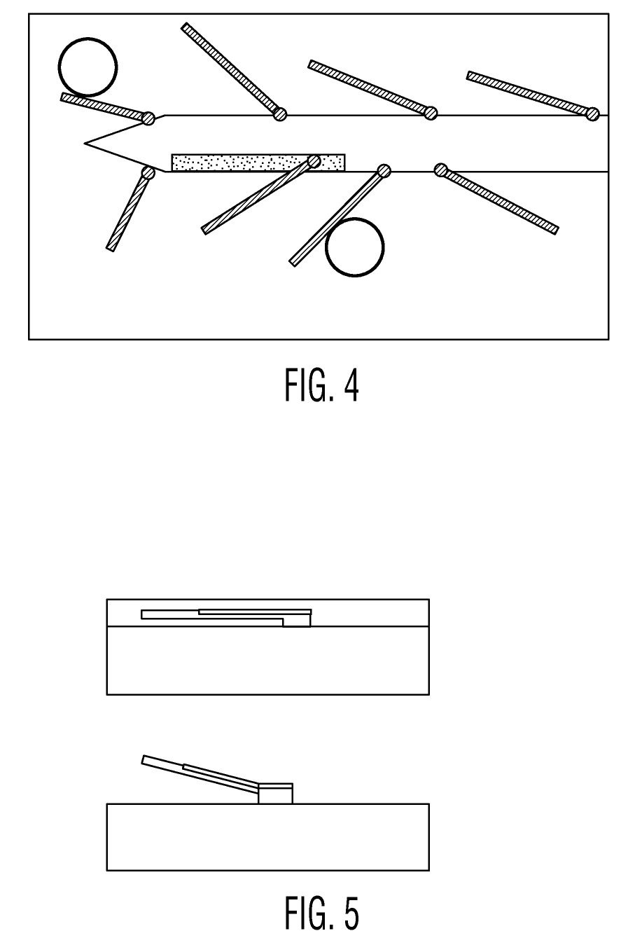

FIG. 4 is a sketch depicting the implantable electrode system according to the invention after implantation into the body.

FIG. 5 is a sketch depicting an embodiment of the invention showing the unreleased and released positions, respectively, of the hinged electrodes relative to the implant body surface both before and after implantation into the body.

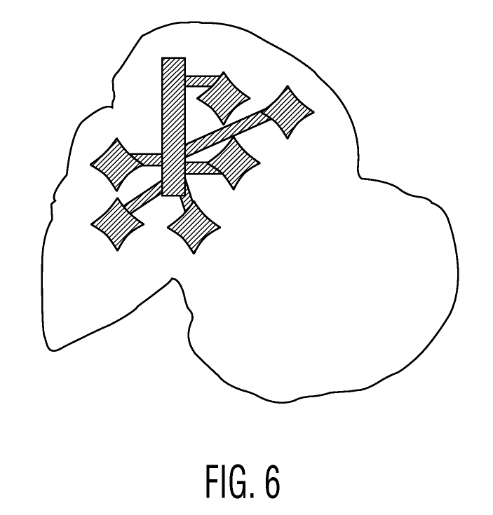

FIG. 6 is a sketch depicting the implant body after implantation and self-assembly of the extended electrode branches within the surrounding neural tissue sites for electrical recording and/or stimulation.

According to the invention an implantable medical system, for example an electrode system, for electrical recording and or providing therapy, for example, drug delivery or stimulation of a plurality of neural tissue sites without damage to surrounding blood vessels in a human or animal body is disclosed.

The system includes a main body with a plurality of therapy elements, for example, electrodes that are completely coated or encased within a coating material, for example, a biodegradable material or frozen water, which after implantation causes slow dissolution of the coating material within the body, permitting a release mechanism to release the electrode into several branches extending out of the main body of the implant to create a “tree”—like 2-dimensional or 3-dimensional structure. The electrode branches are extended slowly after the insertion of the implant, with or without external control, but essentially for the present invention without any damage to blood vessels surrounding the implant. Such electrode system provides the closest interface to the neural tissue with much reduced possibility of insertion damage. By using this approach, significant improvements in selectivity, power consumption and biocompatibility can be achieved, and can be considered as a minimally-invasive approach to electrodes introduction. It is also contemplated within the scope of the invention disclosed herein to utilize known mainstream integrated circuit (IC) manufacturing components and techniques, making it cost-effective. The system and method of the invention can also be extended to any application where electrical coupling to single or multiple cells is used for sensing/stimulation purposes.

FIG. 3 depicts the implantable electrode system according to the invention before implantation into the human or animal body. The electrode branches are attached to the body of the device at one end by hinges that only allow extension of the branches with a given pressure. Branches are held in place by a biodegradable coating material shaped for the easiest insertion of the implanted device.

FIG. 4 depicts the implantable electrode system after the implantation. The biodegradable encapsulation is dissolved, releasing the branches to extend into the surrounding neural tissue. The force during the electrode branches extension should be chosen to be enough to extend into the neural tissues, but lower than the threshold amount to puncture the surrounding blood vessel walls. The surface of the implant body itself can also be made functional. Thus, a vast area of implant-tissue interface is created, with the possibility to access remote parts of the nervous tissue without complicated implantation procedures. Implant can have sufficiently sophisticated electronics to stimulate and sense neural activity at different branches. Both branches and implant body can be functionalized using “ArrayFET” (Field Effect Transistor) technology.

The implantable electrode system can be fabricated, for example, by coating the implant body (made, for example of silicon) with a biodegradable material (for example, Poly(DL-lactide-co-glycolide) (PLGA). The PLGA is a polymer which degrades by hydrolysis [see J. G. Hardy and T. S. Chadwick, Clin. Pharmacokinet. 39, 1-4 (2000)]. The byproducts of hydrolysis of PLGA are glycolic acid and lactic acid. Glycolic acid either is passed in urine or forms glycine which is metabolized by the tricarboxylic acid cycle. Lactic acid is a natural byproduct of muscle contraction and likewise enters the tricarboxylic acid cycle [see K. A. Athanasiou, C. E. Agrawal, F. A. Barber, and S. S. Burkhart, J. Arthrosc. Relat. Surg., 14(7), (1998) 726] is deposited and then patterned to open the holes where the electrodes are hingedly connected to the implant body. A “stress coating material layer” patterned on top of the exposed upper electrode surfaces (as shown in FIG. 5). The entire body and nonextended electrodes coated with stress material is then completely embedded by the biodegradable material by an extra deposition. This “stress” material should have a lower Young's modulus than the electrode material to create a differential stress sufficient to bend the whole electrode outward from the implant body (as shown in the bottom figure of FIG. 5) as soon as the biodegradable material is dissolved in the tissue. The situation can be achieved by combination of, for example, silicon as the electrode material and gold as the “stress” material [see Lijie Li, Justyna Zawadzka, and Deepak Uttamchandani, “Integrated Self-Assembling and holding technique Applied to a 3-D MEMS Variable Optical Attenuator”, Journal of Microelectromechanical Systems, Vol. 13, No. 1, p. 83 (2004)], but should be chosen to create the exact amount of force to move the electrode branches in the tissue but not to perforate the blood vessels. Any other known MEMS (i.e., micro-electro-mechanical systems) technique can be used here too. Alternatively, the electrode branches can be extended after implantation utilizing other methodology than described herein, such as, for example, without self-assembly, but rather using external controller means.

In another embodiment, the coating material may be frozen water. In this case the therapy elements would be folded to the implant body and be frozen in a coating of water prior to implanting into the body of the mammal (human or animal). After insertion into the body of the mammal, the frozen water coating would defrost and melt, releasing the therapy elements from the folded position into the tissue sites.

The suggested invention is exemplified in use for providing therapy by neural tissue interfacing, for example, in an implantable neurostimulation medical device. It can also be extended to any application where electrical coupling to single or multiple cells is used for sensing/stimulation. Additionally, within the framework of the invention disclosed herein, it is contemplated that other materials can be used for the electrodes, the biodegradable coating material and the stress coating material, which would be known to one skilled in the art. Also, within the framework of the invention, it is contemplated that the electrical components of the medical device can be interconnected by electrical wires or wirelessly; thus, for example, in the case of neurostimulation it is contemplated the electrodes can be detached from the rest of the medical device body if needed by, for example, the movement of the surrounding tissue.

In another alternative embodiment of the invention, the systems and methodology can be applied in providing therapy involving drug delivery to tissue sites in the body of a mammal.

While the present invention has been described with respect to specific embodiments thereof, it will be recognized by those of ordinary skill in the art that many modifications, enhancements, and/or changes can be achieved without departing from the spirit and scope of the invention. Therefore, it is manifestly intended that the invention be limited only by the scope of the claims and equivalents thereof.

1. An implantable medical system for providing electrical recording and or therapy to one or more tissue sites of a mammal without damage to surrounding blood vessels comprising:
an implant body having at least one therapy element, each element being hingedly attached at one end to the surface of the body and releasably extendable outward from the surface of the body at the other end;
a release mechanism for each element; and
a coating material covering the body and each element; wherein upon dissolution of the coating material after implantation, the release mechanism is capable of causing each of the elements to extend outward at one end from the surface of the body and into the one or more tissue sites without damage to the surrounding blood vessels.

2. The system of claim 1 wherein at least one of the therapy elements is capable of delivering a drug to the one or more tissue sites.

3. The system of claim 1 wherein the coating material is frozen water.

4. An implantable electrode system according to claim 1 for electrical recording and or stimulation of a plurality of neural tissue sites without damage to surrounding blood vessels comprising:
an implant body having a plurality of electrodes, the electrodes being hingedly attached at one end to the surface of the body and releasably extendable outward from the surface of the body at the other end;
a release mechanism for each of the electrodes; and
a biodegradable coating material covering the body and the electrodes; wherein upon dissolution of the coating material after implantation, the release mechanism is capable of causing the electrodes to extend outward at one end from the surface of the body and into a plurality of neural tissue sites without damage to the surrounding blood vessels.

5. The system of claim 4 wherein the release mechanism comprises a stress coating material on a portion of the outer surface of the electrode, the stress coating material having a lower Young's modulus value than that of the electrode; and
the biodegradable coating material covers the body and the stress coated electrodes.

6. The system of claim 4 wherein the implant body is made of silicon.

7. The system of claim 4 wherein the biodegradable coating material is poly(dl-lactide-co-glycolide) polymer which degrades by hydrolysis.

8. The system of claim 4 wherein the electrodes are made of silicon and the stress coating material is gold.

9. A method of implanting an implantable medical system for electrical recording and or providing therapy to one or more tissue sites without damage to surrounding blood vessels, the method comprising:
implanting the system into a desired location having the tissue sites, the system comprising:
an implant body having at least one therapy element, the element being hingedly attached at one end to the surface of the body and releasably extendable outward from the surface of the body at the other end;
a release mechanism for each element; and
a coating material covering the body and each element; wherein upon dissolution of the coating material after implantation, the release mechanism is capable of causing each of the elements to extend outward at one end from the surface of the body and into one or more tissue sites;
and activating the release mechanism thereby causing each of the elements to extend outwardly at one end from the surface of the body and into the one or more tissue sites without damage to the surrounding blood vessels.

10. The method of claim 9 wherein at least one of the therapy elements is capable of delivering a drug to the one or more tissue sites.

11. The method of claim 9 wherein the coating material is frozen water.

12. The method according to claim 9 of implanting an implantable electrode system for electrical recording and or stimulation of a plurality of neural tissue sites without damage to surrounding blood vessels, the method comprising:
implanting the system into a desired location having the neural tissue sites, the system comprising:
an implant body having a plurality of electrodes, the electrodes being hingedly attached at one end to the surface of the body and releasably extendable outward from the surface of the body at the other end;
a release mechanism for each of the electrodes; and
a biodegradable coating material covering the body and the electrodes; wherein upon dissolution of the coating material after implantation, the release mechanism is capable of causing the electrodes to extend outward at one end from the surface of the body and into a plurality of neural tissue sites;
and activating the release mechanism thereby causing each of the electrodes to extend outwardly at one end from the surface of the body and into the plurality of neural tissue sites without damage to the surrounding blood vessels.

13. The method of claim 12 wherein the release mechanism comprises a stress coating material on a portion of the outer surface of the electrode, the stress coating material having a lower Young's modulus value than that of the electrode; and
the biodegradable coating material covers the body and the stress coated electrodes.

14. The method of claim 12 wherein the implant body is made of silicon.

15. The method of claim 12 wherein the biodegradable coating material is poly (dl-lactide-co-glycolide) polymer which degrades by hydrolysis.

16. The method of claim 12 wherein the electrodes are made of silicon and the stress coating material is gold.
